(19) United States
(12) Patent Application Publication
(10) Pub. No.: US 2026/0273013 A1
(43) Pub. Date: Sep. 17, 2026

(54) COMPOSITION FOR IMPROVING GENETIC STABILITY COMPRISING TEA TREE EXTRACT

(71) Applicant: AMOREPACIFIC CORPORATION, Seoul (KR)

(72) Inventors: Si Young Cho, Yongin-si (KR); A Young Kim, Yongin-si (KR); Hee Young Jeon, Yongin-si (KR); Kyeong Hwan Hwang, Yongin-si (KR); Yu Mi Kang, Yongin-si (KR); Shin Hyoung Kim, Yongin-si (KR); Hyoung June Kim, Yongin-si (KR); Ji Yong Jung, Yongin-si (KR); Yong Deog Hong, Yongin-si (KR)

(73) Assignee: AMOREPACIFIC CORPORATION, Seoul (KR)

(21) Appl. No.: 19/560,707

(22) Filed: Mar. 9, 2026

(30) Foreign Application Priority Data
Mar. 12, 2025 (KR) 10-2025-0032218

(51) Int. Cl. A61K 36/82; A23F 3/16; A61K 8/9789; A61P 17/00; A61Q 19/08

(52) U.S. Cl. CPC A61K 36/82; A23F 3/163; A61K 8/9789; A61P 17/00; A61Q 19/08

(57) ABSTRACT

The present specification relates to a composition comprising a Jangwon No. 3 tea plant extract as an active ingredient. The composition, by comprising the Jangwon No. 3 tea plant extract as the active ingredient, improves gene damage repair capability, and thus can provide an effect of improving genomic stability, which is important for inhibiting epigenetic skin aging. Through this, by improving expression of Col7A1 and Col17A1, which are collagen types known to most affect skin sagging, skin sagging is prevented, and skin sagging can be improved.

### CROSS REFERENCE TO RELATED APPLICATION

The present application claims priority to Korean Patent Application No. 10-2025-0032218, filed Mar. 12, 2025, the entire contents of which are hereby incorporated by this reference.

### BACKGROUND OF THE INVENTION

### Field of the Invention

The present specification describes a composition comprising a tea plant extract.

### Description of the Related Art

Endogenous genotoxic substances such as reactive oxygen species (ROS) or aldehydes and exogenous genotoxic substances such as chemical compounds in the environment, UV, or X-rays induce DNA damage, and such DNA damage induces genomic instability by inhibiting accurate replication, controlled transcription, and safe storage of genetic information. The genomic instability induces death of cells themselves or promotes aging by causing epigenetic changes. The genomic instability itself does not directly affect functionality and performance of the cells, but DNA methyltransferase is present at a damage site during a DNA repair process, and various chromatin remodeling factors perform a role in assembly of various repair mechanisms, thereby forming epigenetic traces after damage removal and restoration of an initial chromatin state. Therefore, the epigenetic traces are formed by continuously occurring DNA damage and repair, and this contributes to epigenetic heterogeneity between cells and decline of age-related gene expression control, thereby inducing epigenetic aging. The genomic instability may be overcome through a DNA repair system. The DNA repair system is decreased during a progression of aging, and although it is being reported that such a decrease in repair capability is closely related to occurrence of many diseases related to aging, until now, a solution of enhancing genomic stability through improvement of the DNA repair system decreased by aging has not been reported.

### SUMMARY OF THE INVENTION

In one aspect, the present disclosure has been made in an effort to provide a composition capable of inhibiting epigenetic skin aging by improving genomic instability.

In one aspect, one embodiment of the present disclosure provides a composition for improving genetic stability, comprising a Jangwon No. 3 tea plant extract as an active ingredient.

The composition according to one embodiment of the present disclosure, by comprising the Jangwon No. 3 tea plant extract among various tea plant varieties as the active ingredient, promotes expression of XPC (Xeroderma pigmentosum complementation group C) and XRCC4 (X-Ray Repair Cross Complementing 4), which are genes essential for a DNA repair process decreased by aging, and thus may provide an effect of improving genomic stability, which is important for inhibiting epigenetic skin aging, by improving gene damage repair capability. Through this, by improving expression of Col7A1 and Col17A1, which are collagen types known to most affect skin sagging, skin sagging can be prevented, or skin sagging can be improved.

### BRIEF DESCRIPTION OF THE DRAWINGS

FIG. 1 is a view comparing a DNA repair system capability recovery effect of Example 1 (JW3) and Comparative Example 1 (GTE) according to one embodiment of the present disclosure through fluorescence images.

FIG. 2 is a view comparing a genetic stability recovery effect against external stress (H₂O₂, etoposide) of Example 1 (JW3) and Comparative Example 1 (GTE) according to one embodiment of the present disclosure through fluorescence images.

FIG. 3 is a view showing a graph comparing the genetic stability recovery effect against external stress (H₂O₂, etoposide) of Example 1 (JW3) and Comparative Example 1 (GTE) according to one embodiment of the present disclosure.

FIG. 4 is a view showing a comparison of an amount of change in beta-galactosidase activity in aging-induced cells treated with Example 1 (JW3) and Comparative Example 1 (GTE) according to one embodiment of the present disclosure.

FIG. 5 is a view showing a comparison of an expression amount of Lamin B1 in aging-induced cells treated with Example 1 (JW3) and Comparative Example 1 (GTE) according to one embodiment of the present disclosure.

FIG. 6 is a view showing a comparison of expression amounts of collagen Col7A1 and Col17A1 in aging-induced cells treated with Example 1 (JW3) and Comparative Example 1 (GTE) according to one embodiment of the present disclosure.

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, constituent elements, components (or parts), or combinations thereof, but do not exclude the presence or addition of one or more other features, integers, steps, operations, constituent elements, components (or parts), or combinations thereof.

One embodiment of the present disclosure may provide a composition for improving genetic stability, comprising a Jangwon No. 3 tea plant extract as an active ingredient.

Another embodiment may provide a use of the Jangwon No. 3 tea plant extract for use in manufacture of a composition for improving genetic stability. Another embodiment may provide a method for improving genetic stability, comprising administering the Jangwon No. 3 tea plant extract in an effective amount to a subject in need thereof. Another embodiment may provide the Jangwon No. 3 tea plant extract as an active ingredient for use in a composition for improving genetic stability. In addition, a non-therapeutic use of the Jangwon No. 3 tea plant extract as an active ingredient for improving genetic stability may be provided.

In the present disclosure, the Jangwon No. 3 is a variety name of a new variety of Camellia sinensis, Theaceae, and a scientific name is Camellia sinensis L. The Jangwon No. 3 exhibits morphological characteristics that young shoots with one bud and three leaves and a length of a leaf blade are on a long side, a posture of the leaf blade is upward, an intensity of a color of the leaf blade is medium, and a position where a flower style splits is high. Compared with Yabukita, which is a variety generally used for a green tea extract, it exhibits morphological differences that a width of a leaf blade is narrow, a shape of the leaf blade is a narrower ellipse, and a serrated shape of a leaf blade edge is deep.

In one embodiment, the Jangwon No. 3 may be KCTC (Korean Collection for Type Cultures) 12213BP deposited on May 14, 2012 with the Korea Research Institute of Bioscience and Biotechnology.

Also, the extract of the Jangwon No. 3 tea plant also exhibits differences in types and contents of constituent components from an extract of a general tea plant variety.

In one embodiment, the extract of the Jangwon No. 3 tea plant may be one obtained by processing buds or leaves of Jangwon No. 3 tea plant.

In one embodiment, the extract of the Jangwon No. 3 tea plant may comprise methylated catechin (epigallocatechin-3-O-(3-O-methyl)-gallate (EGCG3″Me)) in a high content of about 10 times or more than the extract of the general tea plant variety. Specifically, the Jangwon No. 3 tea plant extract may comprise the methylated catechin in 0.5 to 10 wt % relative to a total weight of the extract. More specifically, the Jangwon No. 3 tea plant extract may comprise the methylated catechin in 0.5 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 6 wt % or more, 7 wt % or more, 8 wt % or more, or 9 wt % or more relative to the total weight of the extract, and may be comprised in 10 wt % or less, 9 wt % or less, 8 wt % or less, 7 wt % or less, 6 wt % or less, 5 wt % or less, 4.5 wt % or less, 4 wt % or less, 3.5 wt % or less, 3 wt % or less, 2.5 wt % or less, 2 wt % or less, 1.5 wt % or less, or 1 wt % or less.

In one embodiment, the extract of the Jangwon No. 3 tea plant may further comprise one or more selected from the group consisting of gallocatechin, epigallocatechin, catechin, epicatechin, epigallocatechin gallate, gallocatechin gallate, and epicatechin gallate in addition to the methylated catechin.

In one embodiment, the extract of the Jangwon No. 3 tea plant may comprise 0.5 to 10 wt %, more specifically, 1 to 3 wt % of methylated catechin relative to the total weight of the extract. In one embodiment, the extract of the Jangwon No. 3 tea plant may comprise 0.41 to 1 wt %, more specifically, 0.45 to 0.7 wt % of gallocatechin relative to the total weight of the extract. In one embodiment, the extract of the Jangwon No. 3 tea plant may comprise 8 to 15 wt %, more specifically, 10 to 15 wt % of epigallocatechin relative to the total weight of the extract. In one embodiment, the extract of the Jangwon No. 3 tea plant may comprise 0.3 to 1 wt %, more specifically, 0.4 to 0.6 wt % of catechin relative to the total weight of the extract. In one embodiment, the extract of the Jangwon No. 3 tea plant may comprise 2.1 to 10 wt %, more specifically, 2.5 to 4.5 wt % of epicatechin relative to the total weight of the extract. In one embodiment, the extract of the Jangwon No. 3 tea plant may comprise 16 to 30 wt %, more specifically, 18 to 20 wt % of epigallocatechin gallate relative to the total weight of the extract. In one embodiment, the extract of the Jangwon No. 3 tea plant may comprise 0.11 to 0.5 wt %, more specifically, 0.15 to 0.4 wt % of gallocatechin gallate relative to the total weight of the extract. In one embodiment, the extract of the Jangwon No. 3 tea plant may comprise 3 to 15 wt %, more specifically, 4 to 10 wt % of epicatechin gallate relative to the total weight of the extract.

In one embodiment, the extract of the Jangwon No. 3 tea plant may be one obtained by processing first-flush tea leaves of Jangwon No. 3 tea plant.

Generally, for tea plant, when harvested once, leaves grow from one bud and one leaf to five leaves, and such a harvesting process is repeatedly performed about four times a year.

When the leaves of tea plant are classified according to a harvesting period, these may be named first-flush tea, second-flush tea, third-flush tea, and fourth-flush tea. Specifically, the first-flush tea may be one in which tea leaves are harvested for a first time by May of a corresponding year, the second-flush tea may be one harvested for a second time after harvesting the first-flush tea of the corresponding year (harvested in June to July), the third-flush tea may be one harvested for a third time after harvesting the second-flush tea of the corresponding year (harvested in August), and the fourth-flush tea may be one harvested for a fourth time after harvesting the third-flush tea of the corresponding year (harvested on or after September). Also, when the leaves of tea plant are classified according to leaf growth, they may be divided into a first leaf to a fifth leaf according to an order in which leaves emerge from one tea plant (one bud), and it takes about 30 days to 35 days until becoming the fifth leaf after sprouting.

From the above aspect, the ‘first-flush tea’ according to one embodiment may mean one in which tea leaves are harvested for the first time by May of the corresponding year. The ‘first-flush tea’ according to one embodiment may mean one in which tea leaves are harvested at a one-bud and five-leaf stage. For example, the extract of the Jangwon No. 3 tea plant may be one in which the upper first leaf to third leaf of a new shoot are collected based on a period when an opening degree of the one-bud and five-leaf stage is 70% during a first-flush tea period of the Jangwon No. 3 tea plant. In the present disclosure, general green tea corresponding to the first-flush tea means one using tea leaves collected at remaining periods excluding the first-flush tea.

In one embodiment, the Jangwon No. 3 tea plant extract may be an aqueous ethanol solution extract of Jangwon No. 3 tea plant leaves.

In one embodiment, the Jangwon No. 3 tea plant extract may be one manufactured by an extraction method comprising: a step of steaming the tea leaves by steaming and then drying; a step of pulverizing and powdering a dried product obtained in the step; a step of extracting the powdered tea leaves using an aqueous ethanol solution as an extraction solvent; and a step of filtering an extract obtained in the extraction step and then concentrating under reduced pressure to obtain an extract. In one embodiment, the steaming by steaming may be steaming by passing the tea leaves through a steamer for 20 to 60 seconds. In one embodiment, the concentration under reduced pressure may be performed at 40 to 45° C.

In one embodiment, the composition may comprise the Jangwon No. 3 tea plant extract in 0.0001 to 50 wt % relative to a total weight of the composition. Specifically, the composition may comprise the Jangwon No. 3 tea plant extract in 0.0001 wt % or more, 0.001 wt % or more, 0.01 wt % or more, 0.1 wt % or more, 1 wt % or more, 5 wt % or more, 10 wt % or more, 15 wt % or more, 20 wt % or more, 25 wt % or more, 30 wt % or more, 35 wt % or more, 40 wt % or more, or 45 wt % or more relative to a total weight of the composition, and may comprise the same in 50 wt % or less, 45 wt % or less, 40 wt % or less, 35 wt % or less, 30 wt % or less, 25 wt % or less, 20 wt % or less, 15 wt % or less, 10 wt % or less, 5 wt % or less, 1 wt % or less, or 0.1 wt % or less.

In one embodiment, the Jangwon No. 3 tea plant extract promotes expression of XPC and XRCC4, which are genes essential for a DNA repair process decreased by aging, thereby quickly repairing DNA damage, and thus genetic stability may be improved and epigenetic aging may be inhibited. From the above aspect, the composition according to one embodiment may be for inhibiting epigenetic skin aging.

Another embodiment may provide a use of the Jangwon No. 3 tea plant extract for use in manufacture of a composition for inhibiting epigenetic skin aging. Another embodiment may provide a method for inhibiting epigenetic skin aging, comprising administering the Jangwon No. 3 tea plant extract in an effective amount to a subject in need thereof. Another embodiment may provide the Jangwon No. 3 tea plant extract as an active ingredient for use in a composition for inhibiting epigenetic skin aging. In addition, a non-therapeutic use of the Jangwon No. 3 tea plant extract as an active ingredient for inhibiting epigenetic skin aging may be provided.

Also, in one embodiment, the Jangwon No. 3 tea plant extract may improve skin sagging by improving expression of Col7A1 and Col17A1, which are collagen types known to be important for a dermal epidermal junction (DEJ) function that most affects skin sagging.

From the above aspect, the composition according to one embodiment may be for preventing or improving skin sagging.

Another embodiment may provide a use of the Jangwon No. 3 tea plant extract for use in manufacture of a composition for preventing or improving skin sagging. Another embodiment may provide a method for preventing or improving skin sagging, comprising administering the Jangwon No. 3 tea plant extract in an effective amount to a subject in need thereof. Another embodiment may provide the Jangwon No. 3 tea plant extract as an active ingredient for use in a composition for preventing or improving skin sagging. In addition, a non-therapeutic use of the Jangwon No. 3 tea plant extract as an active ingredient for preventing or improving skin sagging may be provided.

In one embodiment, the composition according to one embodiment of the present disclosure may be a cosmetic composition. The cosmetic composition may be formulated by containing a cosmetologically or dermatologically acceptable medium or base. This refers to all formulations suitable for topical application, and may be for transdermal administration.

For example, it may be provided in a form of a solution, a gel, a solid, a paste anhydrous product, an emulsion obtained by dispersing an oil phase in an aqueous phase, a suspension, a microemulsion, microcapsules, microgranules, or ionic (liposome) and non-ionic vesicle dispersants, or in a form of a cream, a toner, a lotion, a powder, an ointment, a spray, or a concealer stick. Also, it may be used in a form of a foam or in a form of an aerosol composition further comprising a compressed propellant. As another example, it may be a formulation of an injectable such as a filler, a patch, and the like, but is not limited thereto. These compositions may be manufactured according to conventional methods in the art.

As one embodiment, the composition according to one embodiment of the present disclosure, in addition to the active ingredient, may comprise other components capable of preferably giving a synergistic effect to a main effect within a range that does not damage the main effect, and in addition to the active ingredient of the present disclosure, other components may be appropriately selected and blended by those skilled in the art without difficulty according to a formulation or a purpose of use of other cosmetic compositions. Also, as one embodiment, the cosmetic composition of the present disclosure, in addition to the above components, may comprise other components usually blended in a cosmetic composition as necessary. For example, a moisturizer, an emollient, organic and inorganic pigments, organic powder, a UV absorber, a preservative, a bactericide, an antioxidant, a plant extract, a pH adjuster, alcohol, colorants, fragrance, a blood circulation promoter, a cooling agent, an antiperspirant, purified water, and the like may be mentioned. Other blending components capable of being comprised in the cosmetic composition of the present disclosure are not limited thereto, and a blending amount of the components is possible within a range that does not damage the purpose and effect of the present disclosure.

The composition according to one embodiment of the present disclosure may be an oral composition comprising the active ingredient. As one embodiment, the composition may be a food composition. For example, it may be processed into functional foods such as fermented milk, cheese, yogurt, juice, probiotic preparations, and health foods comprising the active ingredient, and may be used in a form of various other food additives. As one embodiment, the composition may be a composition for health food. As one embodiment, the composition for health food may be formulated into a pill, a capsule, a tablet, granules, a caramel, or a drink, or the like. As another embodiment, it may also be processed in a form of a liquid, powder, granules, a tablet, or a tea bag, or the like. The composition may be administered by various methods such as simple drinking, injection administration, a spray method, or a squeeze method. The composition may comprise other components and the like capable of giving a synergistic effect to a main effect within a range that does not damage the main effect of the present disclosure. For example, to improve physical properties, additives such as fragrance, a colorant, a bactericide, an antioxidant, a preservative, a moisturizer, a thickener, inorganic salts, an emulsifier, and a synthetic polymer substance may be further comprised. In addition to that, auxiliary components such as water-soluble vitamins, oil-soluble vitamins, high molecular peptides, high molecular polysaccharides, and seaweed extract may be further comprised. The components may be appropriately selected and blended by those skilled in the art according to a formulation or a purpose of use, and an addition amount thereof may be selected within a range that does not damage the purpose and effect of the present disclosure. For example, the addition amount of the components may be 0.0001 wt % to 99.9 wt % based on a total weight of the composition.

Hereinafter, the present disclosure will be described in detail with reference to Examples, Comparative Examples, and Test Examples. It will be obvious to those having ordinary knowledge in the art that these are merely presented illustratively to describe the present disclosure more specifically, and a scope of the present disclosure is not limited by these Examples, Comparative Examples, and Test Examples.

### [Example 1] Manufacture of Jangwon No. 3 Tea Plant Extract

Jangwon No. 3 (Osulloc Farm Co., Ltd., Agricultural Corporation) was collected from the upper first leaf to third leaf of a new shoot based on a period when an opening degree of a one-bud and five-leaf stage was 70% during a first-flush tea (early May) period, and was steamed by passing through a steamer for 40 seconds, dried in a dryer at 80° C., and then powder-processed by a pulverizer, sieved through a 60-mesh sieve, and powdered. To 100 g of the pulverized sample of the Jangwon No. 3 thus manufactured, 1 L of a 70% (v/v) aqueous ethanol solution (anhydrous ethanol 70% (v/v)+distilled water 30% (v/v)) was added, and after performing reflux extraction and filtering, it was concentrated under reduced pressure at 40 to 45° C., and finally 19.2 g of a dried extract of leaves of a new variety of tea plant (Jangwon No. 3) was secured.

### Comparative Example 1

A green tea extract from a general tea plant was manufactured in the same method as in Example 1, except that a variety of tea plant was Yabukita (Samkyung Costech).

### [Test Example 1] Comparison of Catechin Composition Comprised in Tea Plant Extract

For a comparative analysis of catechin components comprised in the tea plant extracts of Example 1 and Comparative Example 1 manufactured above, the following experiment was performed.

After dissolving each of the extracts in 50% methanol to make a 10,000 ppm solution, component analysis (Waters Co., 2695/2996 PDA detector) was performed using high-performance liquid chromatography (HPLC) (Waters Co.). For a stationary phase, a Thermo Syncronis C18 (4.6×250 mm, 5 μm), P/N: 97105-254630 column was used, and for a mobile phase, a composition ratio as shown in Table 1 below was used.

TABLE 1

| Time | A: 0.1% Acetic | |
|---|---|---|
| (minutes) | acid in water | B: Acetonitrile |
| 0 | 90 | 10 |
| 10.0 | 90 | 10 |
| 30.0 | 85 | 15 |
| 53.0 | 80 | 20 |
| 53.1 | 5 | 95 |
| 55.0 | 5 | 95 |
| 55.1 | 90 | 10 |
| 60.0 | 90 | 10 |

As shown in Table 2 below, it was confirmed that the extract of Jangwon No. 3 leaves comprises epigallocatechin-3-O-(3-O-methyl) gallate (EGCG3″Me; 3″-O-Me-EGCG) in an amount of 2.5 wt % based on a total weight of the extract, which is comprised in a high content of 10 times or more than a general tea plant variety.

TABLE 2

| | | Comparative |
|---|---|---|
| | Example 1 | Example 1 |
| | (Jangwon | (General green |
| Item | No. 3 extract) | tea extract) |
| (1) gallocatechin (GC) | 0.5 | 0.4 |
| (2) epigallocatechin (EGC) | 12.7 | 7.7 |
| (3) catechin (C) | 0.5 | 0.2 |
| (4) epicatechin (EC) | 3.1 | 2.0 |
| (5) epigallocatechin gallate (EGCG) | 18.8 | 15.1 |
| (6) gallocatechin gallate (GCG) | 0.2 | 0.1 |
| (7) epicatechin gallate (ECG) | 5.3 | 2.6 |
| (8) catechin gallate (CG) | N/D | N/D |
| | (Not Detected) | |
| Total content of 8 types of catechins | 41.1 | 28.1 |
| 3″-O-Me-EGCG content | 2.5 | 0.2 |

(wt %)

### [Test Example 2] Confirmation of Expression Promotion Effect of Genes Related to DNA Repair System

To confirm an epigenetic aging inhibitory effect by improvement of genetic stability of the extracts of Example 1 and Comparative Example 1, the following experiment was performed.

First, to induce aging of skin keratinocytes, skin keratinocytes (normal human epidermal keratinocytes, NHEK) were seeded to be 12,000 cells/cm² and cultured until reaching 80% confluency. To the cell culture, UVB 20 mJ/cm² was repeatedly irradiated once a day for 3 days, and cultured for 5 days. In this case, 25 ppm of Jangwon No. 3 extract (Example 1), 25 ppm of general green tea extract (Comparative Example 1), or DMSO (dimethyl sulfoxide, control group) was cultured while being repeatedly treated at each ultraviolet (UVB) irradiation.

The keratinocytes in which aging was induced by repeated ultraviolet irradiation while being treated with the Jangwon No. 3 extract (Example 1), the general green tea extract (Comparative Example 1), or DMSO (control group) were extracted for RNA using an RNeasy mini kit (Qiagen), and after synthesizing cDNA from the extracted RNA using a SuperScript VILO Master Mix (Invitrogen), expression amount changes of XPC (Xeroderma pigmentosum complementation group C, Hs00190295_ml) and XRCC4 (X-Ray Repair Cross Complementing 4, Hs00243327_ml, Applied Biosystems, Foster City, CA, USA) were observed by real-time qPCR (a 7500 Fast Real-time PCR System, Thermo Fisher Scientific, Waltham, MA, USA), and relative expression amounts based on the control group in which the aging-induced cells were treated with DMSO are shown in the table below.

TABLE 3

| | | | Aging | | Aging | | Aging | |
|---|---|---|---|---|---|---|---|---|
| | Control group | | induction | | induction | | induction and | |
| | before aging | | and control | | and | | Comparative | |
| | induction | | group | | Example 1 | | Example 1 | |
| | (Young_con) | | (Old_con) | | (Old_JW3) | | (Old_GTE) | |
| XPC | 2.6 | 0.05 | 1 | 0.002 | 2.1 | 0.004 | 1.2 | 0.01 |
| XRCC4 | 4.2 | 0.02 | 1 | 0.003 | 2.6 | 0.005 | 1.2 | 0.03 |

As a result, as shown in the table above, in the aging-induced cells (Old_con), expression amounts of XPC and XRCC4, which are genes essential for a DNA repair process, were decreased compared with the control group before aging induction (Young_con), and it was confirmed that for the general green tea extract (green tea extract, GTE), the expression amounts of XPC and XRCC4 were hardly recovered even though it is known as an antioxidant. On the other hand, in Example 1 in which the Jangwon No. 3 tea plant extract, which is the active ingredient of the present disclosure, was administered, the expression amounts of XPC and XRCC4 were recovered to a level of the control group before aging induction. This means that the Jangwon No. 3 tea plant extract, which is the active ingredient of the present disclosure, has an excellent genetic stability improvement effect through DNA damage repair capability decreased by aging which does not appear in the general green tea extract, and accordingly, can provide an epigenetic aging inhibitory effect.

### [Test Example 3] Verification of DNA Repair Capability Recovery Effect

For the keratinocytes in which aging was induced by repeated ultraviolet irradiation while being treated with the Jangwon No. 3 extract (Example 1), the general green tea experiment (Comparative Example 1), or DMSO (Dimethyl sulfoxide, control group) obtained in Test Example 2, a DNA repair capability recovery effect was verified as follows.

Specifically, 50 mg/ml of pEGFPN1 plasmid (Addgene, USA) was treated with 20% H₂O₂ for 1 hour to induce DNA damage in order to induce DNA cleavage, so that green fluorescent protein (GFP) is not expressed. This DNA damage-induced plasmid was transfected into keratinocytes using Effectene (Qiagen), and then after 24 hours, fluorescence of GFP was measured using Zen software (Carl Zeiss, Jena, Germany) under a fluorescence microscope (Zeiss LSM700 confocal microscope). A relative GFP fluorescence expression degree (% RE) was calculated as follows.

$$RE = F / Fu \qquad [ Equation 1 ]$$

Here, F=N_{GFP}×MI/N_{LC}, N_{GFP} is GFP-positive cells, MI is GFP fluorescence intensity, N_{LC} is live total cells, and Fu is a result value of transfecting pEGFPN1 that is not treated.

TABLE 4

| | Control | Example 1 | Comparative |
|---|---|---|---|
| | group (DMSO) | (JW3) | Example 1 (GTE) |
| % RE | 3.42 ± 0.7 | 13.5 ± 0.9 | 4.9 ± 0.9 |

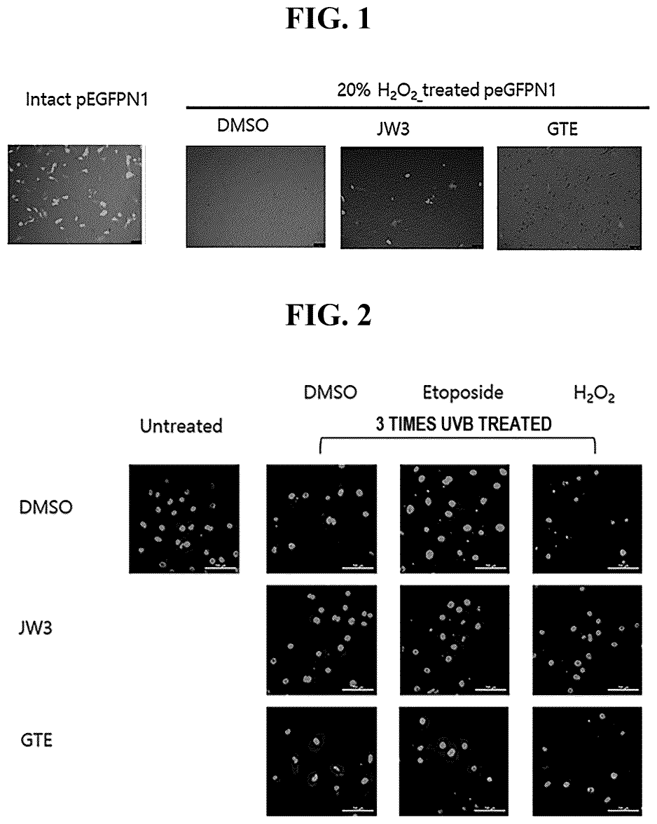

As a result, as shown in Table 4 and FIG. 1, in the pEGFPN1 plasmid treated with Example 1, it appeared that the GFP fluorescence expression amount was increased by about 3 times than the general green tea extract, and thus it can be confirmed that DNA repair system capability was effectively recovered.

### [Test Example 4] Verification of Genetic Stability Recovery Effect Against Various External Stresses

In this experiment, for H₂O₂ and etoposide, which are representative stresses inducing genomic instability by causing DNA damage, a genetic stability recovery effect of the Jangwon No. 3 extract, which is the active ingredient according to one embodiment of the present disclosure, was to be verified.

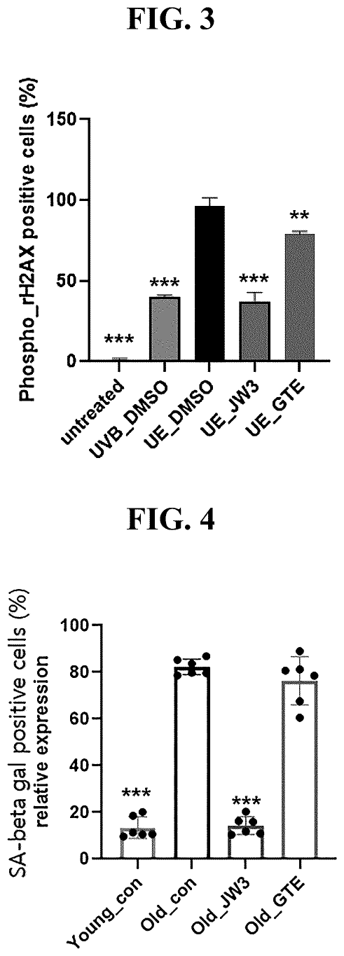

To the keratinocytes in which aging was induced by repeated ultraviolet irradiation while being treated with the Jangwon No. 3 extract (Example 1), the general green tea extract (Comparative Example 1), or DMSO (Dimethyl sulfoxide, control group) obtained in Test Example 2, 100 mM H₂O₂ and 10 mM etoposide were treated for 2 hours, respectively, and then fixed with 3.7% formaldehyde. After reacting Phospho-Histone H2A.X (Cell Signaling, MA, USA) as a primary antibody for 16 hours, a fluorescence-attached secondary antibody (Donkey anti-Mouse IgG (H+L) Highly Cross-Adsorbed Secondary Antibody, Alexa Fluor Plus 488, Invitrogen) was reacted and observed using a fluorescence microscope (Zeiss LSM700 confocal microscope) and is shown in FIG. 2, and a result thereof was analyzed and is shown in FIG. 3.

As a result, cells treated with the general green tea extract (GTE, Comparative Example 1) show green fluorescence of Phospho-Histone H2A.X, which is a DNA damage marker, like the control group (DMSO) treated with H₂O₂ and etoposide inducing genetic instability, whereas cells treated with the Jangwon No. 3 extract (JW3, Example 1) did not show the green fluorescence of Phospho-Histone H2A.X, which is the DNA damage marker, in an untreated group (Untreated) having no stress induction and the control group (DMSO-DMSO). This means that in the case of the cells treated with the Jangwon No. 3 extract (JW3, Example 1), genetic stability was recovered.

### [Test Example 5] Verification of Epigenetic Aging Inhibitory Effect

In this experiment, an epigenetic aging inhibitory effect of the Jangwon No. 3 extract, which is the active ingredient according to one embodiment of the present disclosure, was to be verified using aging-related markers.

### Measurement of Senescence-Associated Beta-2Galactosidase Activity

The keratinocytes in which aging was induced by repeated ultraviolet irradiation while being treated with the Jangwon No. 3 extract (Example 1), the general green tea extract (Comparative Example 1), or DMSO (control group) obtained in Test Example 2 were cultured for 5 days, then washed twice with phosphate-buffered saline (PBS), and then fixed using a 3.7% formaldehyde solution. Thereafter, after washing three times with PBS, an SA-beta gal staining reagent (senescence detection kit, Sigma-Aldrich) was added and reacted at 37° C. for 16 hours.

### Measurement of Expression of Lamin B1

For the keratinocytes in which aging was induced by repeated ultraviolet irradiation while being treated with the Jangwon No. 3 extract (Example 1), the general green tea extract (Comparative Example 1), or DMSO (Dimethyl sulfoxide, control group) obtained in Test Example 2, after 5 days, RNA was extracted using an RNeasy mini kit (Qiagen), and after synthesizing cDNA from the extracted RNA using a SuperScript VILO Master Mix (Invitrogen), expression amount changes of Lamin B1 (HsO1059205_m1, Applied Biosystems, Foster City, CA, USA) were observed by real-time qPCR (a 7500 Fast Real-time PCR System, Thermo Fisher Scientific, Waltham, MA, USA).

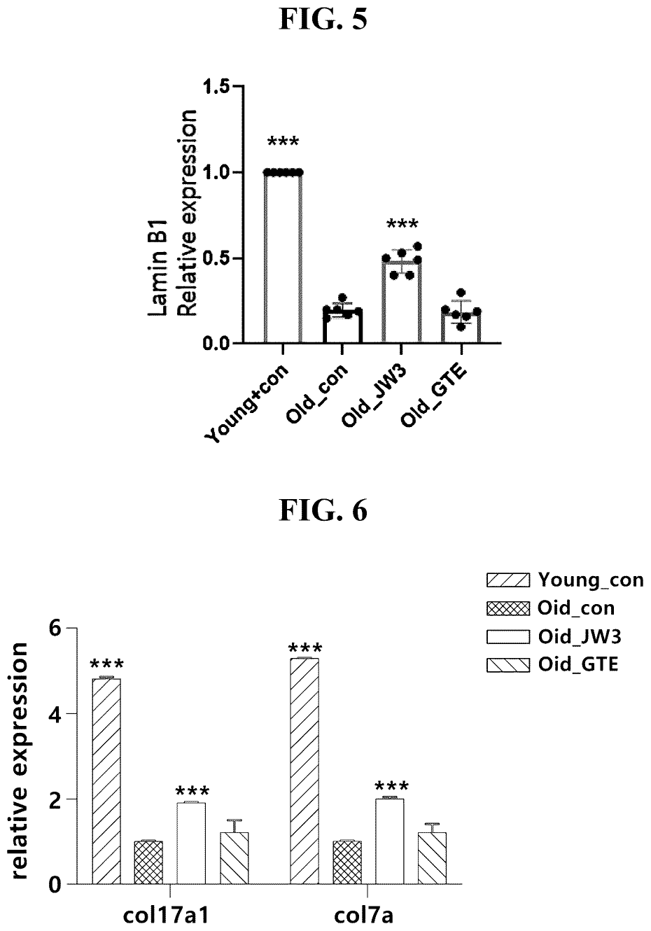

As a result, in the cells treated with the general green tea extract (GTE, Comparative Example 1), almost no change in beta-galactosidase activity appeared, whereas in the cells treated with the Jangwon No. 3 extract (JW3, Example 1), beta-galactosidase activity, which is an aging-related marker, was decreased to a level of the control group (Young_con) in which aging was not induced (FIG. 4). Also, it can be confirmed that an expression amount of Lamin B1 for the Jangwon No. 3 extract (JW3, Example 1) was increased by about 3 times or more than the cells treated with the general green tea extract (GTE, Comparative Example 1) (FIG. 5). This means that the Jangwon No. 3 extract (JW3, Example 1) can provide an epigenetic aging inhibitory effect which does not appear in the general green tea extract.

### [Test Example 6] Verification of Skin Sagging Improvement Effect

In this experiment, through expression amount analysis of Col7A1 and Col17A1, which are collagen types known to be important for a DEJ function important for skin sagging, a prevention of skin sagging and a skin sagging improvement effect of the Jangwon No. 3 extract, which is the active ingredient according to one embodiment of the present disclosure, were to be verified.

Specifically, for the keratinocytes in which aging was induced by repeated ultraviolet irradiation while being treated with the Jangwon No. 3 extract (Example 1), the general green tea extract (Comparative Example 1), or DMSO (Dimethyl sulfoxide, control group) obtained in Test Example 2, after 5 days, RNA was extracted using an RNeasy mini kit (Qiagen), and after synthesizing cDNA from the extracted RNA using a SuperScript VILO Master Mix (Invitrogen), expression amount changes of col7A1 and col17A1 (Hs00164310_ml, Hs00990036_ml, Applied Biosystems, Foster City, CA, USA) were observed by real-time qPCR (a 7500 Fast Real-time PCR System, Thermo Fisher Scientific, Waltham, MA, USA).

As a result, as shown in FIG. 6, it can be confirmed that each expression amount of col7A1 and col17A1 of the Jangwon No. 3 extract (JW3, Example 1) was increased by about 1.5-2 times or more than the cells treated with the general green tea extract (GTE, Comparative Example 1). This means that the Jangwon No. 3 extract (JW3, Example 1) can provide a significantly enhanced effect also on prevention of skin sagging and skin sagging improvement through an epigenetic aging inhibitory effect which does not appear in the general green tea extract.

Formulation Examples of the composition according to one embodiment of the present specification are described below, but application to various other formulations is also possible, and this is not intended to limit the present specification but merely to describe it specifically.

### [Formulation Example 1] Softening Toner (Skin Lotion)

A softening toner was manufactured by a conventional method according to a composition described in the table below.

TABLE 5

| | Content |
|---|---|
| Blending component | (wt %) |
| Jangwon No. 3 tea plant extract | 10 |
| Glycerin | 3.0 |
| Butylene glycol | 2.0 |
| Propylene glycol | 2.0 |
| Carboxyvinyl polymer | 0.1 |
| PEG-12 nonylphenyl ether | 0.2 |
| Polysorbate 80 | 0.4 |
| Ethanol | 10.0 |
| Triethanolamine | 0.1 |
| Preservative, colorant, fragrance | q.s. |
| Purified water | balance |

### [Formulation Example 2] Nourishing Toner (Milk Lotion)

A nourishing toner was manufactured by a conventional method according to a composition described in the table below.

TABLE 6

| | Content |
|---|---|
| Blending component | (wt %) |
| Jangwon No. 3 tea plant extract | 10 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/Capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Preservative, colorant, fragrance | q.s. |
| Purified water | balance |

### [Formulation Example 3] Massage Cream

A massage cream was manufactured by a conventional method according to a composition described in the table below.

TABLE 7

| | Content |
|---|---|
| Blending component | (wt %) |
| Jangwon No. 3 tea plant extract | 10 |
| Glycerin | 8.0 |
| Butylene glycol | 4.0 |
| Liquid paraffin | 45.0 |
| Beta-glucan | 7.0 |
| Carbomer | 0.1 |
| Caprylic/Capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Preservative, colorant, fragrance | q.s. |
| Purified water | balance |

### [Formulation Example 4] Drink

After mixing 50 mg of Jangwon No. 3 tea plant extract, 10 g of glucose, 0.6 g of citric acid, and 25 g of liquid oligosaccharide, 300 ml of purified water is added and each bottle is filled with 200 ml. After filling the bottles, sterilization was performed at 130° C. for 4 to 5 seconds to manufacture a drink.

### [Formulation Example 5] Caramel Formulation

50 mg of Jangwon No. 3 tea plant extract, 1.8 g of corn syrup, 0.5 g of skim milk, 0.5 g of soy lecithin, 0.6 g of butter, 0.4 g of vegetable hydrogenated oil, 1.4 g of sugar, 0.58 g of margarine, and 20 mg of salt were mixed and caramel-molded.

### [Formulation Example 6] Health Food

TABLE 8

| Component | Content | |
|---|---|---|
| Jangwon No. 3 tea | 100 | mg |
| plant extract | | |

Vitamin mixture

| | | |
|---|---|---|
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

A composition ratio of the vitamin and mineral mixture was mixed and composed by taking components relatively suitable for health food as an example, but a blending ratio thereof may be arbitrarily modified and implemented, and after mixing the above components according to a conventional health food manufacturing method, granules are manufactured, and it may be used for manufacture of a health food composition according to a conventional method.

### [Formulation Example 7] Health Drink

TABLE 9

| Component | Content | |
|---|---|---|
| Jangwon No. 3 tea | 10 | mg |
| plant extract | | |
| Citric acid | 1000 | mg |
| Oligosaccharide | 100 | g |
| Plum concentrate | 2 | g |
| Taurine | 1 | g |
| Purified water | balance | |
| Total volume | 900 | ml |

The remaining amount of purified water was added so that a total volume becomes 900 ml as in the table above, and after mixing the above components according to a conventional health drink manufacturing method, and then stirring and heating at 85° C. for about 1 hour, a produced solution is filtered and obtained in a sterilized 2 L container, sealed and sterilized, and then stored under refrigeration to be used for manufacture of a health drink composition.

### Deposit Number

Name of Depositary Institution: Korean Collection for Type Cultures (KCTC), Korea Research Institute of Bioscience and Biotechnology
Deposit number: KCTC12213BP
Deposit date: May 14, 2012

What is claimed is:

1. A method for improving genetic stability, comprising administering a Jangwon No. 3 tea plant extract in an effective amount to a subject in need thereof.

2. The method of claim 1, wherein the extract comprises methylated catechin (epigallocatechin-3-O-(3-O-methyl)-gallate) in 0.5 to 10 wt % relative to a total weight of the extract.

3. The method of claim 2, wherein the extract further comprises one or more selected from the group consisting of gallocatechin, epigallocatechin, catechin, epicatechin, epigallocatechin gallate, gallocatechin gallate, and epicatechin gallate.

4. The method of claim 3, wherein the extract comprises, relative to the total weight of the extract,
0.5 to 10 wt % of methylated catechin;
0.41 to 1 wt % of gallocatechin;
8 to 15 wt % of epigallocatechin;
0.3 to 1 wt % of catechin;
2.1 to 10 wt % of epicatechin;
16 to 30 wt % of epigallocatechin gallate;
0.11 to 0.5 wt % of gallocatechin gallate; and
3 to 15 wt % of epicatechin gallate.

5. The method of claim 1, wherein the Jangwon No. 3 tea plant extract is an aqueous ethanol solution extract of Jangwon No. 3 tea plant leaves.

6. The method of claim 1, wherein the Jangwon No. 3 tea plant extract is comprised in a composition as an active ingredient, and the active ingredient is comprised in 0.0001 to 50 wt % relative to a total weight of the composition.

7. The method of claim 1, wherein the method is for inhibiting epigenetic skin aging through improvement of genetic stability.

8. The method of claim 1, wherein the method is for preventing or improving skin sagging through improvement of genetic stability.

9. The method of claim 1, wherein the Jangwon No. 3 tea plant extract is comprised in a composition as an active ingredient, and the composition is a cosmetic composition.

10. The method of claim 1, wherein the Jangwon No. 3 tea plant extract is comprised in a composition as an active ingredient, and the composition is a food composition.
